(19) United States
(12) Patent Application Publication
(10) Pub. No.: US 2022/0148729 A1
(43) Pub. Date: May 12, 2022

(54) INFORMATION PROCESSING DEVICE

(71) Applicant: NTT DOCOMO, INC., Chiyoda-ku (JP)

(72) Inventors: Naoki YAMAMOTO, Chiyoda-ku (JP); Keiichi OCHIAI, Chiyoda-ku (JP); Takashi HAMATANI, Chiyoda-ku (JP)

(73) Assignee: NTT DOCOMO, INC., Chiyoda-ku (JP)

(21) Appl. No.: 17/434,437

(22) PCT Filed: Jan. 30, 2020
(86) PCT No.: PCT/JP2020/003493

§ 371 Date: Aug. 27, 2021

(30) Foreign Application Priority Data
Mar. 22, 2019 (JP) 2019-055070

(51) Int. Cl. G16H 50/20; A61B 5/16; G06N 20/00

(52) U.S. Cl. CPC G16H 50/20; G06N 20/00; A61B 5/165

(57) ABSTRACT

An object is to provide an information processing device capable of reasonably presenting grounds of an estimation result using a state estimation model. An information processing device (100) includes a model storage unit (104) configured to store a first state estimation model (104a1) for estimating a state of a user on the basis of log information, a second state estimation model (104a2) for estimating a more detailed state when the state of the user is a predetermined state, and a description estimation model (104b) for describing estimation grounds for an estimation result of the first state estimation model (104a1) using log information, a state estimation unit (102) configured to estimate a state of a user using the first state estimation model (104a1) and the second state estimation model (104a2), and a description estimation unit (103) configured to estimate the estimation grounds for the estimation result of the first state estimation model (104a1) on the basis of the description estimation model (104b).

### TECHNICAL FIELD

The present invention relates to an information processing device that determines a state of a user using a learning model.

### BACKGROUND ART

A technology for estimating a stress state of a user on the basis of an operation log of a smartphone or the like is known.

### CITATION LIST

### Patent Literature

[Non-Patent Literature 1] Cognitive Rhythms: Unobtrusive and Continuous Sensing of Alertness Using a Mobile Phone, Saeed Abdullah, Elizabeth Murnane, Mark Matthews, Matthew Kay, Julie Kientz, Geri Gay, Tanzeem Choudhury, Proceedings of the 2016 ACM International Joint Conference on Pervasive and Ubiquitous Computing. ACM, 2016, retrieved on Feb. 6, 2018]

### SUMMARY OF INVENTION

### Technical Problem

Estimation grounds together with an estimation result are required to be shown to a user, but in Patent Literature 1 above, the grounds of estimation of a stress state of a user are unclear.

Therefore, an object of the present invention is to provide an information processing device capable of reasonably presenting the grounds of an estimation result using an estimation model in order to solve the above-described problem.

### Solution to Problem

An information processing device according to the present invention includes a log information storage unit configured to store input data; a model storage unit configured to store a first state estimation model for estimating an output state on the basis of the input data, and a second state estimation model for estimating a more detailed state when the output state is a predetermined state; an estimation processing unit configured to estimate an output state for the input data using the first state estimation model and the second state estimation model; a description model storage unit configured to store a description estimation model for describing estimation grounds for an estimation result of the first state estimation model using the input data; and a description estimation unit configured to estimate the estimation grounds of the estimation result of the first state estimation model on the basis of the description estimation model.

According to the present invention, it is possible to present the output state using the estimation model and to reasonably present the grounds of the estimation result.

### Advantageous Effects of Invention

According to the present invention, it is possible to present the output state using the estimation model and to reasonably present the grounds of the estimation result.

### BRIEF DESCRIPTION OF DRAWINGS

FIG. 1 is a block diagram illustrating a functional configuration of an information processing device 100 that is a model estimation device of the present embodiment.

FIG. 2 is a schematic diagram illustrating an estimation process using a state estimation model 104 and a description estimation model 104b.

FIG. 3 is a flowchart illustrating a state estimation process and a grounds estimation process of the information processing device 100 according to the embodiment.

FIG. 4 illustrates a detailed process of an estimation grounds process of a process S102.

FIG. 5 is a diagram schematically illustrating a SHAP value.

FIG. 6 is a block diagram illustrating a functional configuration of a model construction device 200.

FIG. 7 is a flowchart illustrating a process of constructing each state estimation model.

FIG. 8 is a schematic diagram illustrating a process of generating each state estimation model.

FIG. 9 is a schematic diagram illustrating another example of the process of generating each state estimation model.

FIG. 10 is a diagram illustrating an example of a hardware configuration of an information processing device 100 according to an embodiment of the present disclosure.

### DESCRIPTION OF EMBODIMENTS

Embodiments of the present invention will be described with reference to the accompanying drawings. The same parts are denoted by the same reference signs and repeated description will be omitted, if possible.

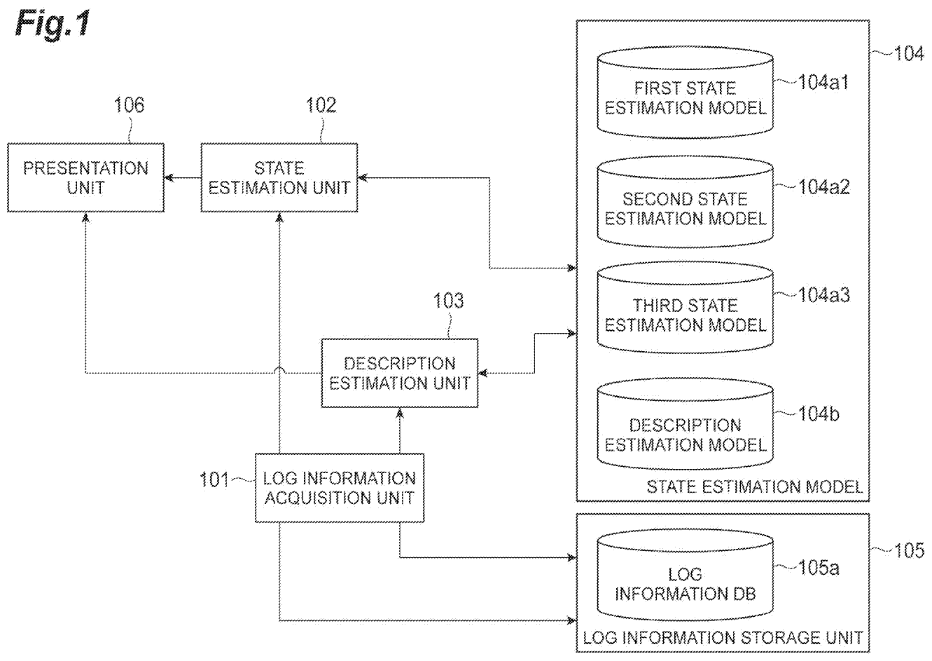

FIG. 1 is a block diagram illustrating a functional configuration of an information processing device 100 that is a model estimation device of this embodiment. As illustrated in FIG. 1, the information processing device 100 includes a log information acquisition unit 101, a state estimation unit 102 (estimation processing unit), a description estimation unit 103 (description estimation unit), a model storage unit 104 (model storage unit or description model storage unit), and a log information storage unit 105. Hereinafter, description will be given with reference to the drawings. The information processing device 100 may be a server located on a network or may be a communication terminal that can be directly operated by a user.

The log information acquisition unit 101 is a unit that acquires log information from a log information DB 105a and converts the log information to a feature quantity.

The state estimation unit 102 is a unit that estimates a state of the user using a state estimation model stored in the model storage unit 104 and the log information stored in the log information storage unit 105.

For example, at least one of an operation history, a movement history, and an access history of a communication terminal such as a smartphone held by the user can be considered as the log information. The state estimation unit 102 can estimate a state of the user such as attention or stress by inputting the log information as a feature quantity or inputting after converting the log information to a predetermined feature quantity format to the state estimation model.

Further, the device is not limited to a device that estimates the state of the user, and may be a device that performs estimation using a learning model for an input.

The description estimation unit 103 is a unit that estimates grounds of the state of the user estimated by the state estimation unit 102 using the log information stored in the log information storage unit and a description estimation model 104b of the model storage unit 104. The description estimation unit 103 can estimate the estimated grounds of the state of the user by receiving the log information input by the log information acquisition unit 101.

The model storage unit 104 is a unit that stores a learning model constructed by machine learning. The model storage unit 104 stores a state estimation model 104a and the description estimation model 104b as learning models.

The state estimation model 104a is, for example, a learning model subjected to a learning process with a feature quantity based on the log information as an explanatory variable and the state of the user as an objective variable. The state estimation model 104a is constructed by a plurality of estimation models. For example, the state estimation model 104a is constructed by a first state estimation model 104a1, a second state estimation model 104a2, and a third state estimation model 104a3.

The state estimation unit 102 estimates a first state or a second state of the user using the first state estimation model 104a1. Depending on a result of the estimation, the state estimation unit 102 estimates a more detailed state of the user using an estimation model that is any one of the second state estimation model 104a2 and the third state estimation model 104a3. For example, when the user is in the first state, the state estimation unit 102 estimates the detailed state of the user using the second state estimation model 104a2.

The description estimation model 104b is a learning model indicating grounds of the state of the user estimated on the basis of the feature quantity based on the log information that is input data. The description estimation model 104b is constructed by SHapley Additive exPlanations (SHAP) or Local Interpretable Model-agnostic Explanations (LIME). SHAP or LIME is a model for calculating an influence of each feature quantity on the prediction. Further, SHAP is a scheme for providing interpretability by approximating a prediction result of a complicated estimation model using a simpler model. The description estimation model 104b is a learning model that is constructed when the state estimation model 104a is constructed. The first state estimation model 104a1 and the description estimation model 104b are models constructed on the basis of the same input data (feature quantity) and the same teacher signal, and the two have a paired relationship.

The log information storage unit 105 stores the log information DB 105a. The log information DB 105 stores, for example, the log information such as an operation history of a communication terminal such as a smartphone held by a user. This log information is information in which time, the operation history, and the like are associated with each other.

A presentation unit 106 is a unit that presents the state of the user (such as the attention and the stress of the user) estimated by the state estimation unit 102 to the user or other persons together with the grounds of the state of the user estimated by the description estimation unit 103.

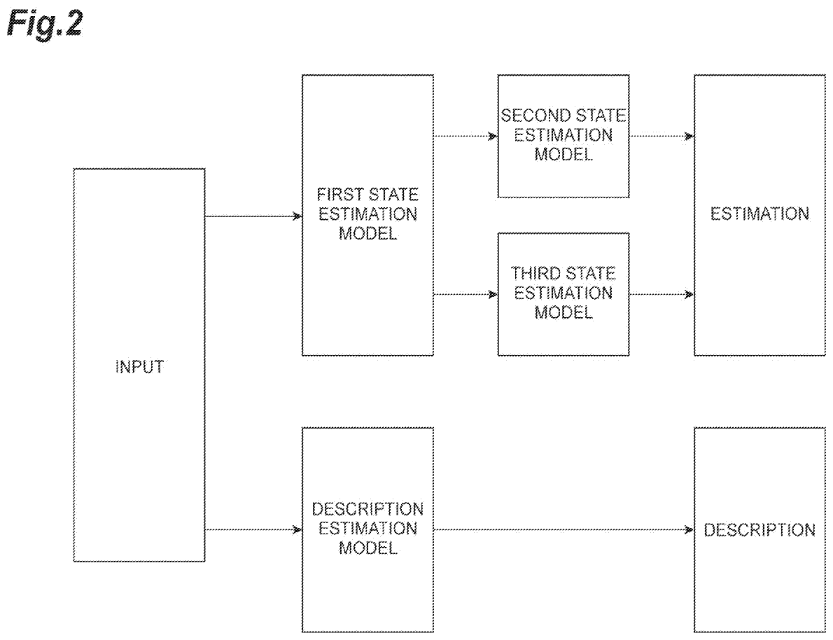

FIG. 2 is a schematic diagram illustrating an estimation process using the first state estimation model 104a1, the second state estimation model 104a2, the third state estimation model 104a3, and the description estimation model 104b. As illustrated in FIG. 2, the first state estimation model 104a1 and the description estimation model 104b estimate the state of the user and its estimation grounds by receiving input data.

In FIG. 2, the first state estimation model 104a1 estimates whether the user is in the first state or the second state with respect to the feature quantity based on the log information. For example, when the first state estimation model 104a1 is a model that is used for estimating the attention of the user, the first state estimation model 104a1 is a model for estimating the first state that is a state in which the user has the attention, and the second state that is a state in which the user has no attention.

The second state estimation model 104a2 is an estimation model that is applied when the state of the user is estimated to be the first state. The second state estimation model 104a2 receives the feature quantity based on the log information input to the first state estimation model 104a1 again and outputs an estimation result on the basis of the feature quantity.

The third state estimation model 104a3 is an estimation model that is applied when the state of the user is estimated to be the second state. The third state estimation model 104a3 receives the feature quantity based on the log information input to the first state estimation model 104a1 again and outputs an estimation result on the basis of the feature quantity.

On the other hand, the description estimation model 104b receives the same feature quantity as the above, and outputs the feature quantity having an influence on a result of the estimation in the first state estimation model 104a1 as a description of the estimation grounds. For example, when the description estimation model 104b is constructed by an SHAP value, the value is output. The SHAP value will be described below.

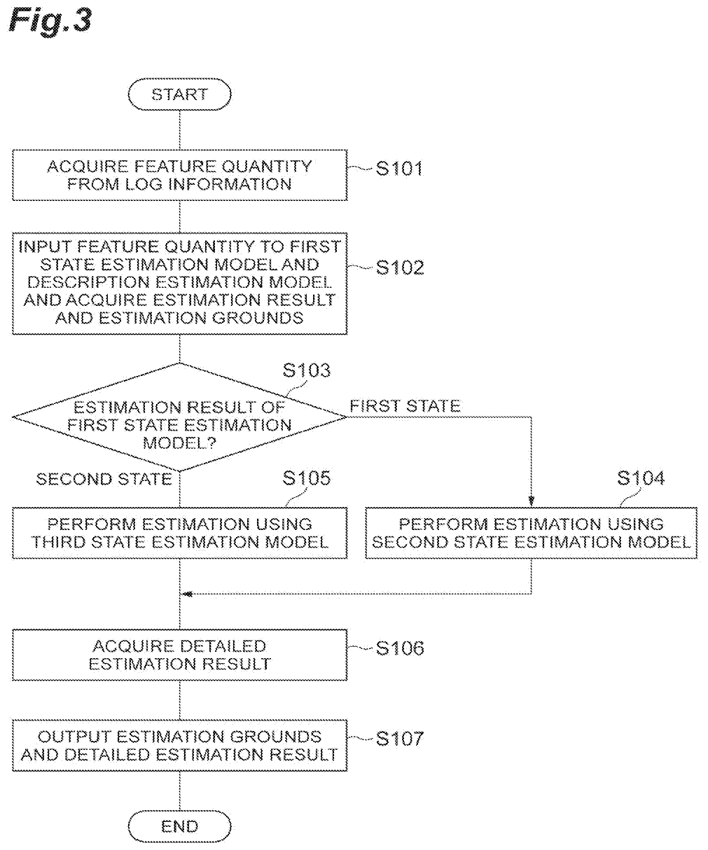

Next, a state estimation process and a grounds estimation process of the information processing device 100 of the embodiment will be described. FIG. 3 is a flowchart illustrating the process.

The log information acquisition unit 101 acquires the log information from the log information DB 105a and converts the log information to a feature quantity (S101). The state estimation unit 102 inputs the feature quantity acquired by the log information acquisition unit 101 to the first state estimation model 104a1 and acquires an estimation result (S102). Further, the description estimation unit 103 inputs the feature quantity acquired by the log information acquisition unit 101 to the description estimation model 104b and estimates a feature quantity (log information) that has influenced the estimation result of the first state estimation model 104a1 (S102). Here, the description estimation unit 103 calculates the SHAP value by inputting the same feature quantity to the description estimation model 104b and estimates the feature quantity that has influenced the estimation result on the basis of the SHAP value. This processing will be described below.

When the state estimation unit 102 estimates that the user is in the first state as the estimation result (S103: YES), the state estimation unit 102 inputs the feature quantity acquired by the log information acquisition unit 101 to the second state estimation model 104a2 again. The state estimation unit 102 performs an estimation process using the second state estimation model 104a2 (S104).

When the state estimation unit 102 estimates that the user is in the first state as the estimation result (S103: NO), the state estimation unit 102 inputs the feature quantity acquired by the log information acquisition unit 101 to the third state estimation model 104a3. The state estimation unit 102 performs the estimation process using the third state estimation model 104a3 (S105).

The state estimation unit 102 acquires the estimation grounds and the estimation result (S106), and the presentation unit 106 outputs the estimation grounds and the estimation result (S107).

Thus, it is possible to show reasonable grounds for the estimation result using the description estimation model 104b and to perform detailed state estimation using the state estimation models 104a1 to 104a3.

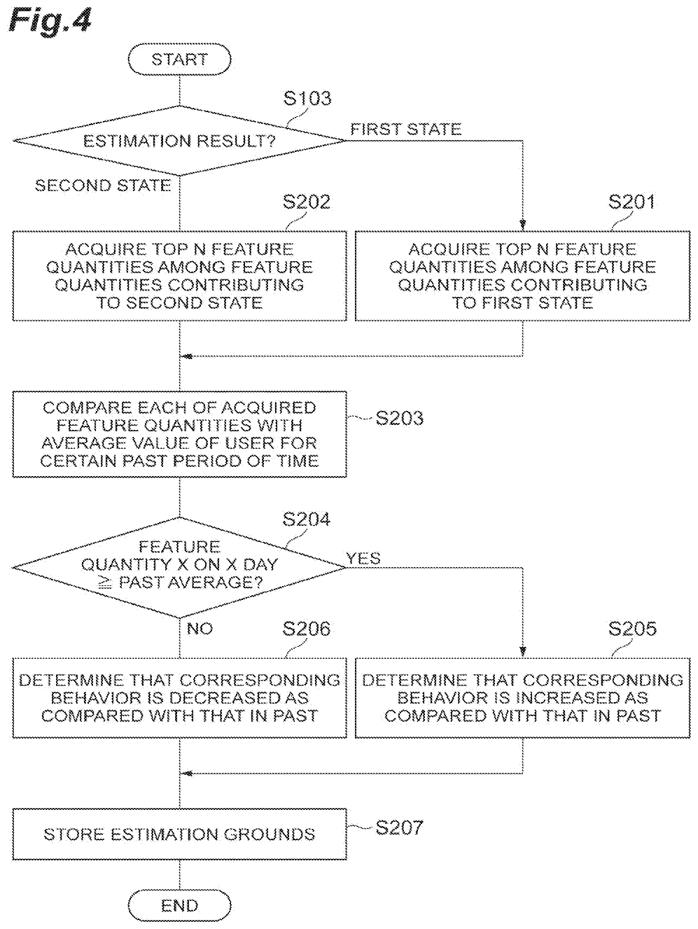

Next, a process of estimating the estimation grounds using the description estimation model 104b will be described. FIG. 4 illustrates detailed processing in the grounds estimation process illustrated in FIG. 3. Specifically, FIG. 4 illustrates detailed processing of the estimation grounds process of the process S102.

The state estimation unit 102 estimates whether the user is in the first state or in the second state by inputting the feature quantity to the state estimation model 104a1 (S103; corresponding to S103 in FIG. 3). When the estimation result is the first state, the description estimation unit 103 acquires the top N feature quantities among the feature quantities contributing to the first state on the basis of the calculated SHAP value (S201). When the estimation result is the second state, the description estimation unit 103 acquires the top N feature quantities among the feature quantities contributing to the second state (S202).

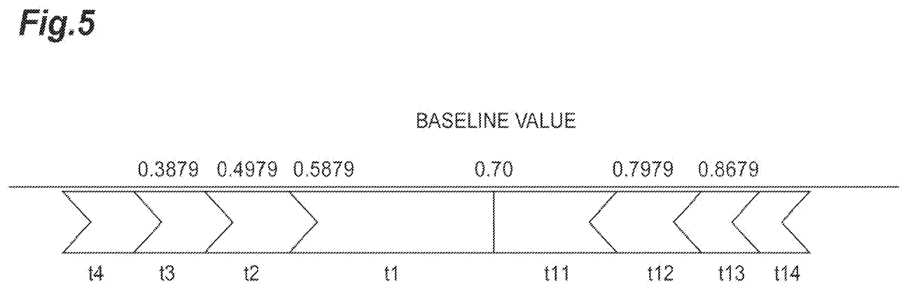

Here, the SHAP value will be explained with reference to the drawings. The SHAP value is a scheme for calculating, for each estimation result, an influence of each feature quantity on the estimation result. FIG. 5 is a diagram schematically illustrating the SHAP value, in which a degree of contribution of each feature quantity is visualized. In FIG. 5, a baseline indicates an expected value of an entire data set. A feature quantity directed to the right from the baseline (a figure arrow: a feature quantity on the left side of the baseline) indicates a positive contribution to the estimated state, and a feature quantity directed to the left (a figure arrow: a feature quantity on the right side of the baseline) indicates a negative contribution to the estimated state. A length of the figure arrow indicates a degree of contribution to the expected value.

For example, a feature quantity t1 has a length of about 0.11, which indicates an increase (contribution) by about 0.11 with respect to the state estimation.

Here, it can be considered that the feature quantities have influenced the estimated state in an order of the length of the arrow, and the top N feature quantities are acquired. Here, the number of estimation grounds that are presented to the user is limited according to the estimation result.

The description estimation unit 103 compares each of the acquired feature quantities with an average value of the user for a certain past period of time (S203). When the acquired feature quantity is equal to or greater than the past average value (S204), the description estimation unit 103 determines that a behavior that is a basis of the feature quantity is increased as compared with that in the past (S205). For example, in an operation with respect to a smartphone, when a feature quantity of a predetermined operation is larger than a past average, a determination is made that the predetermined operation has increased. That is, a determination is made that the behavior has greatly influenced the state estimation.

Further, when the acquired feature quantity is not equal to or greater than the past average value (S204), the description estimation unit 103 determines that the behavior that is the basis of the feature quantity is decreased as compared with that in the past (S206). The description estimation unit 103 temporarily stores these estimation grounds (S207). The stored estimation grounds are output together with the estimation result of the state estimation as illustrated in FIG. 3.

As described above, the number of estimation grounds presented to the user is limited according to the estimation result. When the stress is estimated to be high: 1 in the first state estimation model, only a feature quantity contributing to the estimation of the high stress is presented to the user. On the other hand, when the stress is estimated to be low: 0 in the first state estimation model, only a feature quantity contributing to the estimation of the low stress is presented to the user. In this case, it is possible to inform the user whether a behavior that is grounds for a determination as to a level of the stress is increased or decreased as compared with past behaviors.

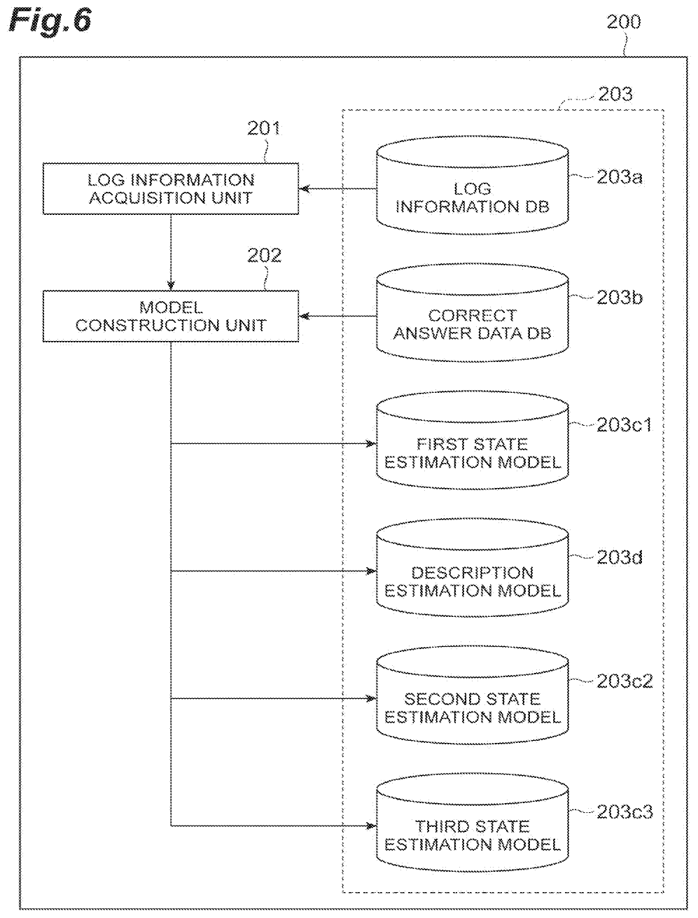

Next, a learning model construction process of the first to third state estimation models 104a1 to 104a3 that realizes such a process will be described. FIG. 6 is a block diagram illustrating a functional configuration of the model construction device 200. The model construction device 200 includes a log information acquisition unit 201, a model construction unit 202, and a storage unit 203, as illustrated in FIG. 6.

The log information acquisition unit 201 is a unit that acquires log information from a log information DB 203a and converts the log information to a feature quantity for model construction.

The model construction unit 202 is a unit that constructs the state estimation model and the description estimation model by performing a learning process using the log information as an explanatory variable and correct answer data as an objective variable.

The storage unit 203 is a unit that stores each database and estimation model, and stores the log information DB 203a, a correct answer data DB 203b, a first state estimation model 203c1, a second state estimation model 203c2, a third state estimation model 203c3, and a description estimation model 203d. The first state estimation model 203c1, the second state estimation model 203c2, the third state estimation model 203c3, and the description estimation model 203d are the state estimation models and the description estimation model constructed by the model construction unit 202, and correspond to the first state estimation model 104a1, the second state estimation model 104a2, the third state estimation model 104a3, and the description estimation model 104b in FIG. 1.

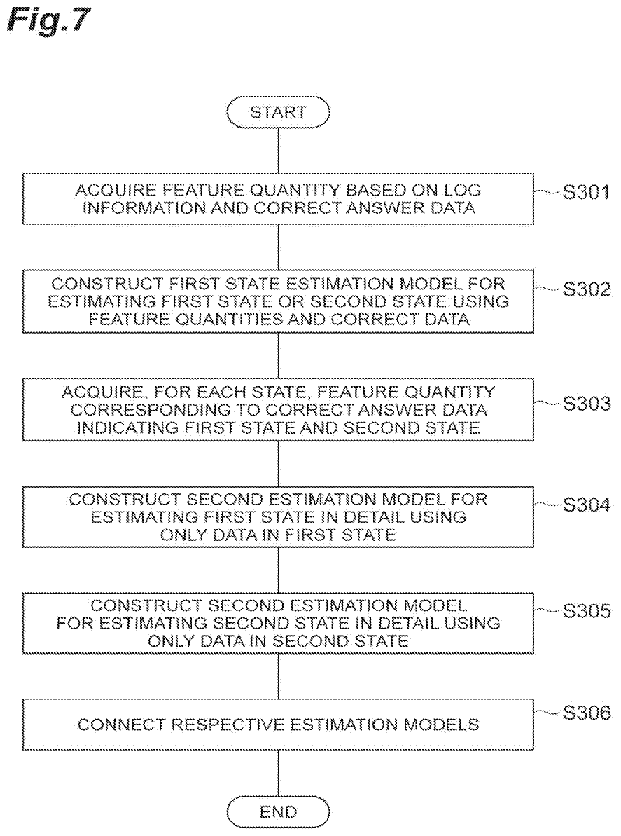

FIG. 7 is a flowchart illustrating a process for constructing each of the state estimation models. The log information acquisition unit 201 acquires the log information from the log information DB 203a, converts the log information to a feature quantity, and inputs the feature quantity to the model construction unit 202. Further, the model construction unit 202 acquires correct answer data from the correct answer data DB 203b (S301).

The correct answer data stored in the correct answer data DB 203b is data prepared in advance. For example, when the correct answer data indicates the attention of the user, the correct answer data is prepared by the user being subjected to a test or the like for measuring the attention in advance. In this case, it is necessary for the correct answer data and the feature quantity (log information) to correspond in time.

The model construction unit 202 constructs the first state estimation model 203c1 for estimating whether the user is in the first state or in the second state using the feature quantity and the correct answer data. The model construction unit 202 performs a learning process using the feature quantity as an explanatory variable and the information obtained by binarizing the correct answer data as an objective variable to construct the first state estimation model (S302). The model construction unit 202 constructs the description estimation model 203d together therewith. In the construction process, a scheme for constructing an estimation model is also used in a well-known description such as SHAD or LIME described above.

Here, the information obtained by binarizing the correct answer data means information obtained by redefining the correct answer data as 1 or 2 according to a predetermined rule. For example, when the correct answer data is represented by five values (1 to 5), 1 and 2 of the correct answer data may be defined as 1 and 3 to 5 may be defined as 2. The binarization process is not limited thereto.

Each function of the model construction device 200 may be included in the information processing device 100.

The model construction unit 202 then acquires correct answer data indicating the first state and the second state and the corresponding feature quantity for each state (S303). That is, the feature quantity estimated to be in the first state and the feature quantity estimated to be in the second state are acquired.

The model construction unit 202 constructs the second state estimation model 203c2 for estimating the first state in detail, using the correct answer data indicating the first state and the feature quantity corresponding thereto (S304). For example, the correct answer data indicating the first state are 1 and 2, and the second state estimation model 203c2 is constructed by performing a learning process using the feature quantity having the correct answer data of 1 and 2.

The model construction unit 202 constructs the third state estimation model 203c3 for estimating the second state in detail, using the correct answer data indicating the second state and the feature quantity corresponding thereto (S305). For example, the correct answer data indicating the second state may be 3 to 5, and the third state estimation model 203c3 may be constructed by performing a learning process using the feature quantity having the correct answer data of 3 to 5.

The model construction unit 202 connects the first to third state estimation models 203c1 to 203c3 (S306). That is, when the first state is estimated in the first state estimation model 203c1, the second state estimation model 203c2 is connected so that the estimation is continued. The same applies to the third state estimation model 203c3. The state estimation unit 102 illustrated in FIG. 1 can apply the state estimation model 104a (203a) according to the state of the user using this connected estimation model.

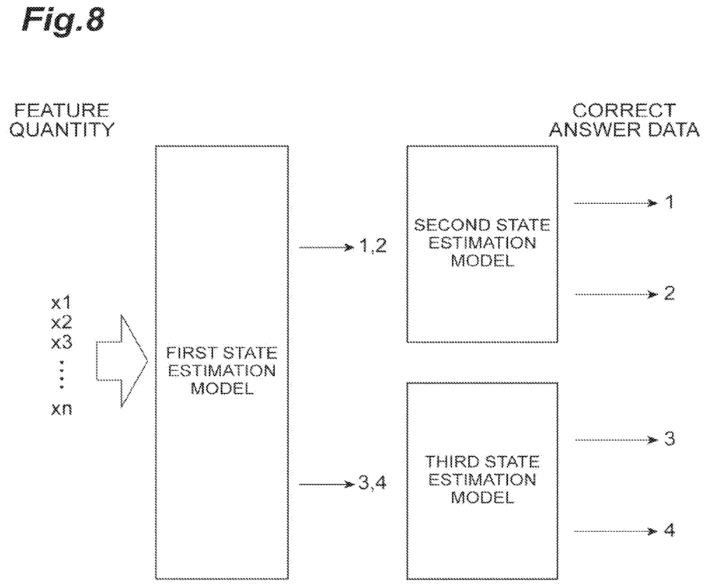

FIG. 8 is a schematic diagram illustrating a process of generating each state estimation model 203a. In this example, 1 to 5 are set as correct answer data, and x1 to xn are set as input feature quantities.

When the first state estimation model 203c1 is generated, the correct answer data corresponding to the feature quantities x1 to xn is replaced with 1 or 2 and the learning process is performed. For example, when the correct answer data corresponding to the feature quantities x11 to x1n is 1, the correct answer data corresponding to the feature quantities x21 to x2n is 2, and the correct answer data corresponding to the feature quantities x31 to x3n is 3, correct answer data of the feature quantities x11 to x1n and the feature quantities x21 to x2n is set to 1, and the correct answer data corresponding to the feature quantities x31 to x3n is set to 2.

That is, the correct answer data 1 and 2 are converted to correct answer data having 1, and the correct answer data 3 to 5 are converted to correct answer data having 2. It is possible to construct the first state estimation model 203c1 by performing a learning process using the converted correct answer data as an objective variable. The first state estimation model 203c1 can output 1 or 2 as correct answer data for the feature quantities x1 to xn.

When the second state estimation model 203c2 is generated, the feature quantities xa1 to xan with the correct answer data set to 1 or 2 are extracted from among the respective feature quantities x11 to x1n and xm1 to xmn. It is possible to construct the second state estimation model 203c2 by performing a learning process in which the extracted feature quantities xa1 to xan are explanatory variables and the correct answer data 1 or 2 corresponding to these feature quantities is an objective variable. The same applies to a process of constructing the third state estimation model 203c3.

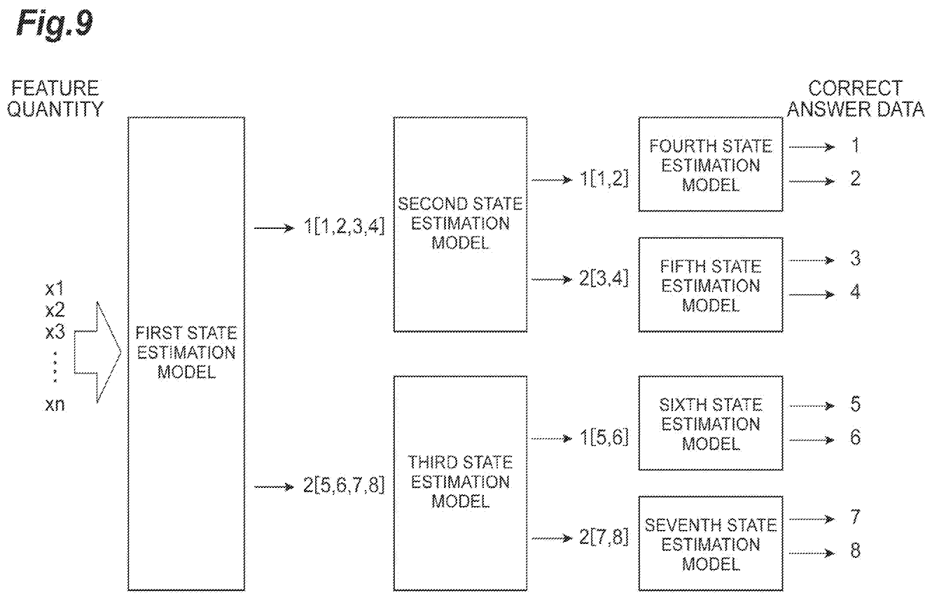

Thus, it is possible to construct the estimation model in stages. The application and the construction of two estimation models connected in series have been described above, but the present invention is not limited thereto. For example, three estimation models may be connected in series to construct an estimation model, and the estimation model may be applied to an estimation process based on feature quantities, as illustrated in FIG. 9. According to this example, 1 to 4 of the correct answer data are replaced with 1 and 5 to 8 of the correct answer data are replaced with 2 to construct the first state estimation model.

Next, operations and effects of the information processing device 100 of the embodiment will be described. The information processing device 100 includes the log information storage unit 105 that stores the log information DB 105a of the log information that is input data, the model storage unit 104 that stores the first state estimation model 104a1 for estimating the state of the user corresponding to the output state on the basis of the feature quantity of the log information, the second state estimation model 104a2 for estimating the more detailed state when the state of the user is a predetermined state (for example, a state in which the user has attention), and the description estimation model 104b for describing the estimation grounds for the estimation result of the first state estimation model 104a1, using the feature quantity based on the log information, the state estimation unit 102 that estimates the state of the user with respect to the log information using the first state estimation model 104a1 and the second state estimation model 104a2, and the description estimation unit 103 that estimates the estimation grounds of the estimation result of the first state estimation model 104a1 on the basis of the description estimation model 104b.

With the information processing device 100, it is possible to estimate the state of the user using the first state estimation model 104a1 and estimate grounds of the estimation. Further, it is possible to estimate the more detailed state of the user using the second state estimation model 104a2. Therefore, it is possible to estimate the state of the user in greater detail, and to easily interpret the grounds for the estimation of the state of the user. Using a plurality of estimation models, it is possible to reduce processing load of a control unit such as a CPU and improve a processing speed of the control unit.

More specifically, by connecting the state estimation models 104a in series, it is possible to perform state estimation using the first state estimation model 104a1 in the previous stage and to perform more detailed state estimation using the second state estimation model 104a2 in the subsequent stage. On the other hand, the description estimation model 104b clarifies the estimation grounds for the state estimated in the previous stage.

The description estimation model 104b is a model that is constructed together when the state estimation model 104a1 is constructed, and both have a correspondence relationship. For example, when the first state estimation model 104a1 is a binary estimation model, the description estimation model 104b is also a binary estimation model. Further, when the first state estimation model 104a1 is a ternary estimation model, the description estimation model 104b is also a ternary estimation model. Therefore, the state estimation and estimation granularity of the estimation grounds thereof are the same.

It is difficult to ascertain detailed grounds of the state and importance is not found there in many cases in performing the state estimation. For example, when the attention of the user is evaluated using five values, it is possible to estimate a case in which the attention is 5 and a case in which the attention is 4. In this case, it is difficult to accurately interpret grounds for estimation that the attention is 4 and the attention is 5.

When the attention of the user is different between 4 and 5, log information thereof is similar and diverse. Therefore, when the description estimation model 104b is applied, descriptions become similar, and it is difficult for the user to understand such grounds for the estimation or a difference. On the other hand, it is relatively easy to estimate the grounds for the estimation in a case in which the attention of the user is 1 and a case in which the attention of the user is 5, and the grounds for estimation are often important. That is, when the attention is 1 and 5, the log information is often different and a difference in the description becomes great.

Therefore, in the information processing device 100 of the embodiment, the estimation models are connected in series to form a two-stage configuration, so that the second state estimation model 104a2 is further connected after the first state estimation model 104a1 in the first stage, as described above. Thus, granularity of state estimation can be made finer, and the description estimation model 104b enables estimation of the estimation grounds according to the estimation granularity of the first state estimation model 104a1. Therefore, it is possible to easily ascertain the estimation grounds and treat the estimation grounds as important ones.

On the other hand, when only the first state estimation model 104a1 is used, it is necessary to perform more detailed state estimation because the granularity of the state estimation is coarse.

In the above description, the input data is the log information and the estimation result based on the input data is the state of the user, but the present invention is not limited thereto. The embodiment can be applied to a process in which there is some input data and estimation is performed on the basis of the input data. Further, it is not always necessary to convert the log information to the feature quantity.

Further, in the information processing device 100, the model storage unit 104 further stores the third state estimation model 104a3. The first state estimation model 104a1 is a learning model for estimating whether the state of the user for the log information is the first state or the second state, and the second state estimation model 104a2 is a learning model for determining the more detailed state when the state of the user is the first state. Further, the third state estimation model 104a3 is a learning model for determining the more detailed state when the state of the user is the second state.

With this information processing device 100, when the first state estimation model 104a1 is a model that performs the binary estimation process, the description estimation model 104b corresponding thereto can also perform the description estimation based on the binary estimation process. Therefore, it is possible to provide the estimation grounds for the state estimation in a form in which the estimation grounds are easy to ascertain.

Further, in the information processing device 100, the description estimation unit 103 indicates one or a plurality of feature quantities that have influenced the state of the user as the estimation grounds of the estimation result. This facilitates ascertaining the estimation grounds thereof.

Further, in the information processing device 100, the description estimation unit 103 acquires the feature quantity serving as the estimation grounds from the description estimation model 104b, and analyzes the estimation result and the log information on the basis of a change in the log information from the past.

With the information processing device 100, it is possible to provide a specific analysis result such that a behavior of the user indicated by the log information is increased or decreased as compared with a past behavior.

Further, the model construction device 200 constructs the first state estimation model 203c1 on the basis of the feature quantity and the correct answer data prepared in correspondence to the log information, and constructs the second state estimation model 203c2 on the basis of the log information used for construction of the first state estimation model 104a1, the correct answer data corresponding to the predetermined state indicating that the second state estimation model 203c2 is applied among the correct answer data corresponding to the log information, and the log information corresponding to the correct answer data.

The information processing device 100 uses the first state estimation model 203c1 and the like constructed in this way as the first state estimation model 104a1 and the like.

Thus, it is possible to construct an appropriate state estimation model according to that state.

There is also a case in which the log information is not converted to the feature quantity, and in this case, the description of the feature quantity may be read as the log information in the above.

The block diagrams used in the description of the embodiment show blocks in units of functions. These functional blocks (components) are realized in any combination of at least one of hardware and software. Further, a method of realizing each functional block is not particularly limited. That is, each functional block may be realized using one physically or logically coupled device, or may be realized by connecting two or more physically or logically separated devices directly or indirectly (for example, using a wired scheme, a wireless scheme, or the like) and using such a plurality of devices. The functional block may be realized by combining the one device or the plurality of devices with software.

The functions include judging, deciding, determining, calculating, computing, processing, deriving, investigating, searching, confirming, receiving, transmitting, outputting, accessing, resolving, selecting, choosing, establishing, comparing, assuming, expecting, regarding, broadcasting, notifying, communicating, forwarding, configuring, reconfiguring, allocating, mapping, assigning, or the like, but the present invention is not limited thereto. For example, a functional block (a component) that functions for transmission is referred to as a transmitting unit or a transmitter. In any case, a realizing method is not particularly limited, as described above.

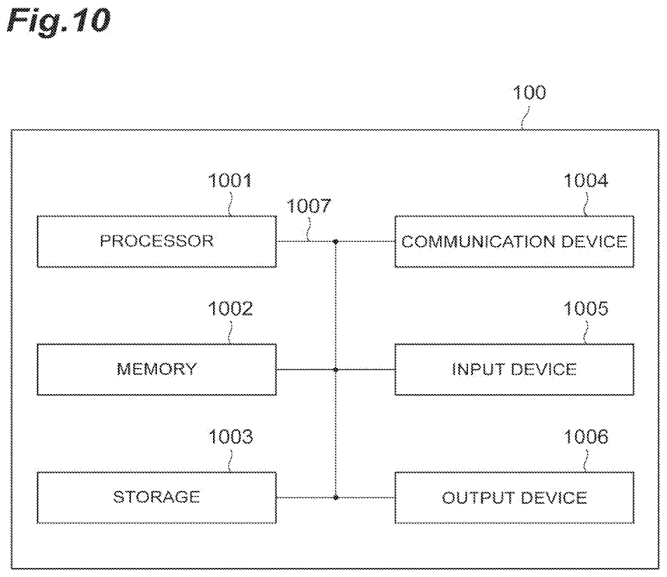

For example, the information processing device 100 and the model construction device 200 according to the embodiment of the present invention may function as a computer that performs processes of an estimation model execution method and an estimation model construction method of the present disclosure. FIG. 10 is a diagram illustrating an example of a hardware configuration of the information processing device 100 and the model construction device 200 according to the embodiment of the present disclosure. The information processing device 100 and the model construction device 200 described above may be physically configured as a computer device including a processor 1001, a memory 1002, a storage 1003, a communication device 1004, an input device 1005, an output device 1006, a bus 1007, and the like.

In the following description, the term “device” can be referred to as a circuit, a device, a unit, or the like. The hardware configuration of the information processing device 100 and the model construction device 200 may include one or a plurality of devices illustrated in FIG. 10, or may be configured without including some of the devices.

Each function in the information processing device 100 and the model construction device 200 is realized by loading predetermined software (a program) into hardware such as the processor 1001 or the memory 1002 so that the processor 1001 performs computation to control communication that is performed by the communication device 1004 or control at least one of reading and writing of data in the memory 1002 and the storage 1003.

The processor 1001, for example, operates an operating system to control the entire computer. The processor 1001 may be configured as a central processing unit (CPU) including an interface with peripheral devices, a control device, a computation device, a register, and the like. For example, the state estimation unit 102, the description estimation unit 103, and the like described above may be realized by the processor 1001.

Further, the processor 1001 reads a program (program code), a software module, data, or the like from at one of the storage 1003 and the communication device 1004 into the memory 1002 and executes various processes according to the program, the software module, the data, or the like. As the program, a program for causing the computer to execute at least some of the operations described in the above-described embodiment may be used. For example, the state estimation unit 102 and the like may be realized by a control program that is stored in the memory 1002 and operated on the processor 1001, and other functional blocks may be realized similarly. Although the case in which the various processes described above are executed by one processor 1001 has been described, the processes may be executed simultaneously or sequentially by two or more processors 1001. The processor 1001 may be realized using one or more chips. The program may be transmitted from a network via an electric communication line.

The memory 1002 is a computer-readable recording medium and may be configured of, for example, at least one of a read only memory (ROM), an erasable programmable ROM (EPROM), an electrically erasable programmable ROM (EEPROM), and a random access memory (RAM). The memory 1002 may be referred to as a register, a cache, a main memory (a main storage device), or the like. The memory 1002 can store an executable program (program code), software modules, and the like in order to implement the state estimation method and the estimation model construction method according to the embodiment of the present disclosure.

The storage 1003 is a computer-readable recording medium and may also be configured of, for example, at least one of an optical disc such as a compact disc ROM (CD-ROM), a hard disk drive, a flexible disc, a magneto-optical disc (for example, a compact disc, a digital versatile disc, or a Blu-ray (registered trademark) disc), a smart card, a flash memory (for example, a card, a stick, or a key drive), a floppy (registered trademark) disk, a magnetic strip, and the like. The storage 1003 may be referred to as an auxiliary storage device. The storage medium described above may be, for example, a database including at least one of the memory 1002 and the storage 1003, a server, or another appropriate medium.

The communication device 1004 is hardware (a transmission and reception device) for performing communication between computers via at least one of a wired network and a wireless network and is also referred to as a network device, a network controller, a network card, or a communication module, for example. The communication device 1004 may include a high-frequency switch, a duplexer, a filter, a frequency synthesizer, and the like, for example, in order to realize at least one of frequency division duplex (FDD) and time division duplex (TDD). For example, the log information acquisition unit 101 described above may be realized by the communication device 1004. Further, a transmission and reception function may be physically or logically separated in implementation.

The input device 1005 is an input device (for example, a keyboard, a mouse, a microphone, a switch, a button, or a sensor) that receives an input from the outside. The output device 1006 is an output device (for example, a display, a speaker, or an LED lamp) that performs output to the outside. The input device 1005 and the output device 1006 may have an integrated configuration (for example, a touch panel).

Further, the respective devices such as the processor 1001 and the memory 1002 are connected by the bus 1007 for information communication. The bus 1007 may be configured using a single bus or may be configured using buses different for each device.

Further, the information processing device 100 and the model construction device 200 may include hardware such as a microprocessor, a digital signal processor (DSP), an application specific integrated circuit (ASIC), a programmable logic device (PLD), or a field programmable gate array (FPGA), and some or all of the functional blocks may be realized by the hardware. For example, the processor 1001 may be implemented by at least one of these pieces of hardware.

Notification of information is not limited to the aspect and embodiment described in the present disclosure and may be made by another method. For example, notification of information may be made by physical layer signaling (for example, downlink control information (DCI) or uplink control information (UCI)), upper layer signaling (for example, radio resource control (RRC) signaling, medium access control (MAC) signaling, or annunciation information (master information block (MIB) or system information block (SIB))), another signal, or a combination of them. Further, RRC signaling may be called an RRC message, and may be, for example, an RRC connection setup message or an RRC connection reconfiguration message.

Further, each aspect/embodiment described in the present disclosure may be applied to at least one of Long Term Evolution (LTE), LTE Advanced (LTE-A), SUPER 3G, IMT-Advanced, 4th generation mobile communication system (4G), 5th generation mobile communication system (5G), Future Radio Access (FRA), new Radio (NR), W-CDMA (registered trademark), GSM (registered trademark), CDMA2000, Ultra Mobile Broadband (UMB), IEEE 802.11 (Wi-Fi (registered trademark)), IEEE 802.16 (WiMAX (registered trademark)), IEEE 802.20, UWB (Ultra Wide Band), Bluetooth (registered trademark), a system using another appropriate system, and a next generation system extended on the basis of such a system. Further, a plurality of systems may be combined (for example, a combination of at least one of LTE and LTE-A, and 5G) and applied.

A process procedure, a sequence, a flowchart, and the like in each aspect/embodiment described in the present disclosure may be in a different order unless inconsistency arises. For example, for the method described in the present disclosure, elements of various steps are presented in an exemplified order, and the elements are not limited to the presented specific order.

Information or the like can be output from an upper layer (or a lower layer) to the lower layer (or the upper layer). The information or the like may be input and output through a plurality of network nodes.

Input or output information or the like may be stored in a specific place (for example, a memory) or may be managed in a management table. Information or the like to be input or output can be overwritten, updated, or additionally written. Output information or the like may be deleted. Input information or the like may be transmitted to another device.

A determination may be performed using a value (0 or 1) represented by one bit, may be performed using a Boolean value (true or false), or may be performed through a numerical value comparison (for example, comparison with a predetermined value).

Each aspect/embodiment described in the present disclosure may be used alone, may be used in combination, or may be used by being switched according to the execution. Further, a notification of predetermined information (for example, a notification of “being X”) is not limited to be made explicitly, and may be made implicitly (for example, a notification of the predetermined information is not made).

Although the present disclosure has been described above in detail, it is obvious to those skilled in the art that the present disclosure is not limited to the embodiments described in the present disclosure. The present disclosure can be implemented as modified and changed aspects without departing from the spirit and scope of the present disclosure defined by the description of the claims. Therefore, the description of the present disclosure is intended for exemplification, and does not have any restrictive meaning with respect to the present disclosure.

Software should be construed widely so that the software means an instruction, an instruction set, a code, a code segment, a program code, a program, a sub-program, a software module, an application, a software application, a software package, a routine, a sub-routine, an object, an executable file, a thread of execution, a procedure, a function, and the like regardless whether the software is called software, firmware, middleware, microcode, or hardware description language or called other names.

Further, software, instructions, information, and the like may be transmitted and received via a transmission medium. For example, when software is transmitted from a website, a server, or another remote source using wired technology (a coaxial cable, an optical fiber cable, a twisted pair, a digital subscriber line (DSL), or the like) and wireless technology (infrared rays, microwaves, or the like), at least one of the wired technology and the wireless technology is included in a definition of the transmission medium.

The information, signals, and the like described in the present disclosure may be represented using any of various different technologies. For example, data, an instruction, a command, information, a signal, a bit, a symbol, a chip, and the like that can be referred to throughout the above description may be represented by a voltage, a current, an electromagnetic wave, a magnetic field or a magnetic particle, an optical field or a photon, or an arbitrary combination of them.

Terms described in the present disclosure and terms necessary for understanding of the present disclosure may be replaced with terms having the same or similar meanings. For example, at least one of a channel and a symbol may be a signal (signaling). Further, a signal may be a message. Further, a component carrier (CC) may be referred to as a carrier frequency, a cell, a frequency carrier, or the like.

The terms “system” and “network” used in the present disclosure are used interchangeably.

Further, the information, parameters, and the like described in the present disclosure may be expressed using an absolute value, may be expressed using a relative value from a predetermined value, or may be expressed using another corresponding information. For example, wireless resources may be indicated by an index.

Names used for the above-described parameters are not limiting names in any way. Further, equations or the like using these parameters may be different from those explicitly disclosed in the present disclosure. Since various channels (for example, PUCCH and PDCCH) and information elements can be identified by any suitable names, the various names assigned to these various channels and information elements are not limiting names in any way.

The term “determining” used in the present disclosure may include a variety of operations. The “determining” can include, for example, regarding judging, calculating, computing, processing, deriving, investigating, search (looking up, search, or inquiry) (for example, search in a table, a database, or another data structure), or ascertaining as “determining.” Further, “determining” can include regarding receiving (for example, receiving information), transmitting (for example, transmitting information), inputting, outputting, or accessing (for example, accessing data in a memory) as “determining.” Further, “determining” can include regarding resolving, selecting, choosing, establishing, comparing or the like as “determining.” That is, “determining” can include regarding a certain operation as “determining.” Further, “determining” may be read as “assuming”, “expecting”, “considering”, or the like.

The terms “connected”, “coupled”, or any modification thereof means any direct or indirect connection or coupling between two or more elements, and can include the presence of one or more intermediate elements between two elements “connected” or “coupled” to each other. The coupling or connection between elements may be physical, may be logical, or may be a combination thereof. For example, “connection” may be read as “access.” When used in the present disclosure, two elements can be considered to be “connected” or “coupled” to each other by using one or more wires, cables, and/or printed electrical connections, or by using electromagnetic energy such as electromagnetic energy having wavelengths in a radio frequency region, a microwave region, and a light (both visible and invisible) region as some non-limiting and non-comprehensive examples.

The description “based on” used in the present disclosure does not mean “based only on” unless otherwise noted. In other words, the description “based on” means both of “based only on” and “based at least on.”

When “include”, “including” and modification of them are used in the present disclosure, these terms are intended to be comprehensive like the term “comprising.” Further, the term “or” used in the present disclosure is intended not to be exclusive OR.

In the present disclosure, for example, when articles such as a, an, and the in English are added by translation, the present disclosure may include that nouns following these articles are plural.

In the present disclosure, a sentence “A and B differ” may mean that “A and B are different from each other.” The sentence may mean that “each of A and B is different from C.” Terms such as “separate”, “coupled”, and the like may also be interpreted, similar to “different.”

### REFERENCE SIGNS LIST

100: Information processing device

101: Log information acquisition unit

102: State estimation unit

103: Description estimation unit

104: Model storage unit

104a: State estimation model

104a1: First state estimation model

104a2: Second state estimation model

104a3: Third state estimation model

104b: Description estimation model

105: Log information storage unit

106: Presentation unit

200: Model construction device

201: Log information acquisition unit

202: Model construction unit

203: Storage unit

203a: State estimation model

203c1: First state estimation model

203c2: Second state estimation model

203c3: Third state estimation model

203d: Description estimation model

1: An information processing device comprising:
a log information storage unit configured to store input data;
a model storage unit configured to store a first state estimation model for estimating an output state on the basis of the input data, and a second state estimation model for estimating a more detailed state when the output state is a predetermined state;
an estimation processing unit configured to estimate an output state for the input data using the first state estimation model and the second state estimation model;
a description model storage unit configured to store a description estimation model for describing estimation grounds for an estimation result of the first state estimation model using the input data; and
a description estimation unit configured to estimate the estimation grounds of the estimation result of the first state estimation model on the basis of the description estimation model.

2: The information processing device according to claim 1,
wherein the model storage unit further stores a third state estimation model,
the first state estimation model is a learning model for estimating that an output state for the input data is a first state or a second state,
the second state estimation model is a learning model for determining a more detailed state when the output state is the first state, and
the third state estimation model is a learning model for determining a more detailed state when the output state is the second state.

3: The information processing device according to claim 1, wherein the description estimation unit indicates one or a plurality of pieces of input data that have influenced the output state, as the estimation grounds of the estimation result.

4: The information processing device according to claim 1, wherein the description estimation unit acquires input data serving as estimation grounds from the description model storage unit, and performs analysis on the estimation result and the input data on the basis of a change in the input data from a past.

5: The information processing device according to claim 1,
wherein the first state estimation model is constructed on the basis of the input data and correct answer data prepared in correspondence to the input data, and
the second state estimation model is constructed on the basis of correct answer data corresponding to a predetermined state indicating that the second state estimation model is applied, and the input data corresponding to the correct answer data among the input data and the correct answer data corresponding to the input data used to construct the first state estimation model.

6: The information processing device according to claim 2,
wherein the first state estimation model is constructed on the basis of the input data and correct answer data prepared in correspondence to the input data, and
the second state estimation model is constructed on the basis of correct answer data corresponding to a predetermined state indicating that the second state estimation model is applied, and the input data corresponding to the correct answer data among the input data and the correct answer data corresponding to the input data used to construct the first state estimation model.
